(19) United States
(12) Patent Application Publication
(10) Pub. No.: US 2026/0284157 A1
(43) Pub. Date: Sep. 24, 2026

(54) POWDER COMPOSITION COMPRISING A HAEMOSTATIC AGENT

(71) Applicants: WOMED, MONTPELLIER (FR); CENTRE NATIONAL DE LA RECHERCHE SCIENTIFIQUE, PARIS (FR); UNIVERSITE DE MONTPELLIER, MONTPELLIER (FR); ECOLE NATIONALE SUPERIEURE DE CHIMIE DE MONTPELLIER, MONTPELLIER CEDEX 5 (FR)

(72) Inventors: XAVIER GARRIC, MONTPELLIER (FR); GONZAGUE ISSENMANN, PARIS (FR); ELODIE GATOUILLAT, MONTPELLIER (FR); MARIA FERRAND, ANIANE (FR)

(21) Appl. No.: 19/471,775

(22) PCT Filed: Apr. 3, 2024
(86) PCT No.: PCT/EP2024/059030

§ 371 Date: Oct. 2, 2025

(30) Foreign Application Priority Data
Apr. 4, 2023 (EP) 23305492.3

(51) Int. Cl. A61K 38/48; A61K 9/16; A61K 31/718; A61K 31/734; A61K 33/14; A61P 7/04

(52) U.S. Cl. CPC A61K 38/4833; A61K 9/1611; A61K 9/1617; A61K 9/1623; A61K 9/1641; A61K 9/1647; A61K 9/1652; A61K 31/718; A61K 31/734; A61K 33/14; A61P 7/04; C12Y 304/21005

(57) ABSTRACT

The present invention relates to a powder composition comprising a specific degradable A and B block copolymer, at least one lubricant, and at least one haemostatic agent. The powder composition according to the invention is particularly useful in a method of preventing and/or treating haemorrhage preferably uterine haemorrhage. The invention also relates to a kit comprising (i) the powder composition of the invention, and (ii) means for inserting said powder composition into a body cavity, preferably into the uterine cavity.

### TECHNICAL FIELD

The invention relates to a powder composition comprising a haemostatic agent and the use of said powder composition in a method of preventing and/or treating haemorrhage, preferably uterine haemorrhage.

### TECHNOLOGICAL BACKGROUND

Acute severe genital haemorrhage (or uterine haemorrhage), of uterine origin and unrelated to pregnancy, is excessive or prolonged bleeding of sufficient volume to require urgent intervention. It is common to classify such uterine bleeding according to its occurrence relative to menstruation. Menorrhagia is upper genital bleeding which coincides with menstruation but is abnormal as regards its abundance or duration. Metrorrhagia denotes any upper genital bleeding which occurs outside of menstruation. It may notably be caused by a pathology of the endometrium or myometrium (hyperplasia, cancer, polyp, fibroid, adenomyosis). Very heavy menorrhagia is an emergency problem and is considered as accidental metrorrhagia. Menometrorrhagia (i.e. the combination of menorrhagia and metrorrhagia) is bleeding from the uterus which is not caused by a tumour, an infection or pregnancy.

It is a common clinical problem and a source of distress for patients as it may be life-threatening. Uterine haemorrhage is, in fact, one of the main causes of maternal mortality. It is thus desirable to control such bleeding as soon as it arises.

The usual treatments for uterine haemorrhage in the emergency room are, in the first instance, venous injections of high-dose conjugated oestrogen, in order to regenerate the endometrium, covering the bare areas at the origin of the bleeding. Intravenous oestrogen alone can stop the bleeding but only five hours after the first administration. Conjugated oestrogens may cause nausea and vomiting. This is the only treatment specifically approved by the FDA for the treatment of acute severe uterine haemorrhage. Antifibrinolytics may also be prescribed and used, and are effective when given in time, within 3 hours of the onset of bleeding. The haemostatic effect generally occurs within 2-3 hours of administration. A rare but serious side effect is the possibility of secondary venous thrombosis. Another approach consists in performing tamponade haemostasis with devices that are usually in the form of a balloon. These devices are invasive and may be left in place for several hours, requiring the patient to remain in bed in the emergency room throughout the treatment. Removal of the balloon requires a further medical intervention for the patient.

There is thus a real need for an intrauterine system that can be easily introduced in the uterine cavity and which allows the rapid and homogeneous release of a haemostatic agent.

### SUMMARY OF THE INVENTION

In this context, the inventors have discovered that a haemostatic agent may be advantageously delivered into a body cavity, in particular into the uterine cavity, by using as a vehicle copolymers in the form of a powder and based on blocks of polyesters, such as polylactic acid (PLA), and blocks of high molecular weight poly(oxyethylene) (PEO). Specifically, such powder copolymers make it possible to produce a material combining dispersion, blood absorption and resorption properties that are particularly suitable for use in the uterine cavity to treat uterine bleeding. The inventors have thus developed a powder composition comprising a haemostatic agent that is easy to introduce into a body cavity such as the uterine cavity, has a good and homogeneous dispersion and advantageously release the haemostatic agent directly onto the body cavity wall, in particular the uterine wall. The powder composition of the invention can cover the entire body cavity, in particular uterine cavity (especially the walls of the body cavity) and maintain haemostasis for a minimum of 24 hours. In addition, the powder composition of the invention does not adhere to the surrounding tissue and does not stimulate cell proliferation.

Compared to a similar system in the form of a film, the powder composition of the invention has the advantages to allow a better coverage of the body cavity, in particular the uterine cavity, because the particles, and thus the haemostatic agent, can be dispersed everywhere. As shown by the experimental data recited in the example section, the powder also allows a faster clotting than with a film. Such a powder composition is further suitable for all shapes and sizes of body cavities and is easily injectable into body cavities. More particularly, such powder composition is suitable for all shapes and sizes of uterus and is easily injectable into the uterine cavity.

The powder composition according to the invention can allow a very rapid release of the haemostatic agent from the moment it is administered into the body cavity, in particular the uterine cavity. Specifically, the release of the haemostatic agent can start directly after administering the system into the body cavity, in particular the uterine cavity. Notably, more than 60% of the haemostatic agent initially present in the material, for example, can be released within 5 minutes of its administration. Furthermore, the material of the powder composition preferably has anti-adhesion properties and the walls of the uterus are then kept separate by the intrauterine system, so that the cicatrization that follows haemostasis does not generate intrauterine adhesions or synechiae. Finally, the disintegration and evacuation time of the intrauterine system according to the invention is generally between 1 and 30 days, which not only allows the intrauterine system to remain in the uterine cavity for a sufficient period of time to treat the bleeding, but also to ensure that it is eliminated naturally, in particular before or during the next menstrual cycle.

One object of the invention is thus a powder composition comprising:
a degradable A and B block copolymer, wherein:
   the A block is a polyester;
   the B block is a poly(oxyethylene) (PEO);
   the weight-average molecular mass of the blocks B is greater than or equal to 50 kDa; and
   the ethylene oxide unit/ester unit mole ratio is between 0.5 and 5;
at least one lubricant; and
at least one haemostatic agent.

Another object is the powder composition of the invention for the administration of the haemostatic agent into a body cavity, preferably into the uterine cavity.

Another object is the powder composition of the invention for use in a method of preventing and/or treating haemorrhage, preferably uterine haemorrhage.

Another object is the powder composition of the invention for use in a method of preventing and/or treating haemorrhage, wherein the composition is for an administration into the body cavity of a subject.

Another object is the powder composition of the invention for use in a method of preventing and/or treating uterine haemorrhage, wherein the composition is for an administration into the uterine cavity of a subject.

Another object of the invention is a kit comprising:
(i) the powder composition of the invention, preferably in a content from 500 mg to 1000 mg; and
(ii) means for inserting said powder into a body cavity, preferably into the uterine cavity.

### BRIEF DESCRIPTION OF THE FIGURES

FIG. 1 presents microscopic observations of powder triblock ABA, dry (left image) and 10 minutes after water contact (right image), in order to demonstrate its swelling properties. Images taken with a Leica microscope, objective ×4. Scale 1000 μm.

FIG. 2 presents microscopic observations of powder triblock ABA, dry (left image) and 30 seconds after water contact (right image), in order to demonstrate its swelling properties. Images taken with a Leica microscope, objective ×10. Scale 400 μm.

FIG. 3 represents the results of an in-vitro release test, where the percentage of thrombin released by the powder composition in function of time has been measured using ELISA method.

In FIG. 4 is presented the clotting time of different prototypes, obtained after an in-vitro clotting test. The prototypes include powder with several thrombin amounts and similar systems in the form of films.

FIG. 5 and FIG. 6 present the results of in-vitro hemostasis tests: different powder compositions are compared based on their clotting time.

In FIG. 7 and FIG. 8 are presented the results of in-vivo studies (wound created on the pig liver), where the bleeding grade of the wound is observed in function of time. Efficacy of different prototypes of powder is evaluated.

In FIG. 9 is presented the clotting time of prototypes G0 to G6, obtained after an in-vitro clotting test. Prototypes include powder with different lubricants mixed with triblock and calcium alginate.

### DETAILED DESCRIPTION

The inventors have developed a powder composition comprising a haemostatic agent which has mechanical and chemical properties that are particularly suitable for its use in the medical field, and in particular for the treatment of haemorrhage such as uterine haemorrhage. Specifically, the dispersion and coverage properties of the copolymers used for preparing the powder composition, combined with the haemostatic agent, make it possible to use it to treat haemorrhage, such as uterine haemorrhage, reliably and rapidly.

### Definitions

In the context of the invention, the expression “between x and y” means that the values x and y are included.

In the context of the invention, the terms “molecular mass” and “molecular weight” are used interchangeably to refer to the weight average molecular mass (Mw), unless otherwise stated. According to the invention, the Mw is determined by size exclusion chromatography performed in dimethylformamide as the analytical solvent, using a poly(ethylene glycol) calibration range.

According to the invention, an “aqueous medium” refers to a medium having an osmolarity similar to the osmolarity of biological fluids. Use is commonly made, as aqueous medium, of phosphate-buffered saline (PBS) considered to be representative of biological fluids.

According to the invention, a “humid medium” refers to a medium equivalent to the aqueous medium, i.e. a medium having an osmolarity similar to the osmolarity of biological fluids, but the humid medium is not liquid. The uterine cavity can be characterized as a non-liquid humid medium.

### The Powder Composition

One subject of the present invention is a powder composition comprising:
a degradable A and B block copolymer, wherein:
   the A block is a polyester;
   the B block is a poly(oxyethylene) (PEO);
   the weight-average molecular mass of the blocks B is greater than or equal to 50 kDa; and
   the ethylene oxide unit/ester unit mole ratio is between 0.5 and 5;
at least one lubricant; and
at least one haemostatic agent.

According to the invention, the term “polyester” denotes any polymer wherein the repeat units of the main chain contain the ester function and which can be used in the medical field. Notably, polyesters is understood to mean aliphatic polyesters such as poly(lactic acid) (PLA), poly(glycolic acid) (PGA), polycaprolactone (PCL), poly(lactic-co-glycolic acid) (PLGA), polybutyrolactone (PBL), polyhydroxyalkanoates (PHA), and copolymers thereof.

In one preferred embodiment, the polyester (A block) is chosen from poly(lactic acid) (PLA), poly(glycolic acid) (PGA), polycaprolactone (PCL) and copolymers thereof. Preferably, the polyester of the A block is chosen from PLA and PCL.

Preferentially, the polyester is in a non-crosslinked form.

The poly(lactic acid) (PLA) may be poly(L-lactic acid), poly(D-lactic acid) or poly(D,L-lactic acid). Advantageously, use is made of poly(D,L-lactic acid) (PDLLA). In this case, the polymer preferentially comprises at least 50 mol % of L-lactic acid, and may particularly comprise at least 60%, 70%, 75%, 80%, 85%, 90%, 95% or 99% of L-lactic acid. Specifically, by modifying the percentage of L-lactic acid relative to the D-lactic acid, it is possible to modify the rate of degradation of the A and B block copolymer. An increase in the level of L-lactic acid makes it possible to slow down the degradation rate of the copolymer. In certain embodiments of the invention, the composition comprises 100% of PLLA as A blocks.

In the context of the invention, the poly(oxyethylene) (PEO) is typically a linear polyether produced from ethylene oxide or ethylene glycol monomers, preferably ethylene oxide monomers. Thus, according to the invention, the B block may also be a polyethylene glycol (PEG) having a high molecular weight greater than or equal to 50 kDa, notably having a molecular weight as defined below.

According to the invention, the poly(oxyethylene) (PEO) used for the B block has a high molecular weight, so that the total molecular weight of the PEO in the copolymer is greater than or equal to 50 kDa.

Advantageously, the total molecular weight of the PEO in the A and B block copolymer is between 50 kDa and 300 kDa. For example, the PEO blocks have a molecular weight of 50 kDa, 75 kDa, 80 kDa, 85 kDa, 90 kDa, 95 kDa, 100 kDa, 105 kDa, 110 kDa, 115 kDa, 120 kDa, 125 kDa, 150 kDa, 200 kDa, 225 kDa, 250 kDa, 275 kDa or 300 kDa. In one particular embodiment, the PEO blocks used have a molecular weight of between 75 kDa and 150 kDa, preferentially between 80 kDa and 125 kDa, more preferentially between 90 kDa and 115 kDa, more preferably between 90 kDa and 110 kDa. In one particular embodiment, the PEO blocks used have a molecular weight of between 95 kDa and 105 kDa.

According to the invention, the PEO block used in the A and B block copolymer advantageously has an inherent viscosity of between 0.04 mg/ml and 0.6 mg/ml, preferentially between 0.08 mg/ml and 0.5 mg/ml, and more preferably between 0.1 mg/ml and 0.3 mg/ml when it is measured by an Ubbelohde type capillary viscometer at a concentration of 1 g/l, at 25° C. in chloroform.

Advantageously, the A and B block copolymer is chosen from AB diblock copolymers, or ABA or BAB triblock copolymers, or mixtures thereof, notably [ABA and BAB], [AB and ABA], [AB and BAB], [ABA and BAB and AB]. In one preferred embodiment, the A and B block copolymers are selected from ABA or BAB triblock copolymers, and preferentially ABA triblock copolymers.

According to the invention, in an AB and/or ABA copolymer, each PEO block (B block) has a molecular weight greater than or equal to 50 kDa and advantageously between 50 kDa and 300 kDa, preferentially between 75 kDa and 150 kDa, preferably between 80 kDa and 125 kDa, more preferentially between 90 kDa and 115 kDa, more preferably between 90 kDa and 110 kDa, or even between 95 kDa and 105 kDa;

whilst in a BAB copolymer, the sum of the molecular weights of the PEO blocks in said copolymer is greater than or equal to 50 kDa and advantageously between 50 kDa and 300 kDa, preferentially between 75 kDa and 150 kDa, preferably between 80 kDa and 125 kDa, more preferentially between 90 kDa and 115 kDa, more preferably between 90 kDa and 110 kDa, or even between 95 kDa and 105 kDa.

In the context of the invention, the molar ratio represents the molar ratio of each of the repeating units (or units) of blocks A and B. As block B is PEO, the repeating units are ethylene oxides (“ethylene oxide unit” or EO), while the repeating units of block A (“ester unit”) are carboxylic acids such as lactic acid units. According to the invention, the EO/ester unit mole ratio in the degradable A and B block copolymer is between 0.5 and 5, and preferably between 1 and 3. The mole ratio is measured from the proton NMR (Nuclear Magnetic Resonance) spectrum in deuterated chloroform of the copolymer, in which the chemical shifts of the characteristic peaks of the PLA-PEO-PLA copolymers may be identified: CH (PLA): 5.1 ppm; CH₂ (PEO): 3.5 ppm; CH₃ (PLA): 1.5 ppm). According to the invention, controlling the EO/LA ratio makes it possible to control the dispersion and blood absorption properties of the powder composition, and also the degradation time. Typically, the lower the EO/LA ratio, the longer the degradation time.

In a specific embodiment of the invention, the EO/ester unit mole ratio in the degradable A and B block copolymer is between 0.5 and 3, or between 0.5 and 2, or between 0.5 and 1.6, or between 0.8 and 3, or between 0.8 and 2, or between 0.8 and 1.6, or between 1 and 3, or between 1 and 2, or between 1 and 1.6.

In a particular embodiment, the degradable A and B block copolymer consists of ABA triblock copolymers, where block A is PDLLA and block B is PEO with a molecular weight of between 90 kDa and 110 kDa, wherein the EO/LA mole ratio is between 0.8 and 2.

In a particular embodiment, the degradable A and B block copolymer consists of ABA triblock copolymers, where block A is PDLLA and block B is PEO with a molecular weight of between 90 kDa and 110 kDa, wherein the EO/LA mole ratio is 1.56.

In a particular embodiment, the degradable A and B block copolymer consists of ABA triblock copolymers, where block A is PDLLA and block B is PEO with a molecular weight of between 90 kDa and 110 kDa, wherein the EO/LA mole ratio is 1.

In a particular embodiment, the degradable A and B block copolymer consists of ABA triblock copolymers, where block A is PDLLA and block B is PEO with a molecular weight of between 90 kDa and 110 kDa, wherein the EO/LA mole ratio is 2.

In another particular embodiment, the degradable A and B block copolymer consists of ABA triblock copolymers, wherein block A is PDLLA and block B is PEO with a molecular weight of between 90 kDa and 110 kDa, wherein the EO/LA mole ratio is 3.

In the context of the invention, the degradable A and B block copolymer is in the form of a powder, preferably with a particle size from 50 μm to 500 μm, more preferably from 150 μm to 500 μm, measured by sieving. According to the invention, controlling the particle size of the degradable A and B block copolymer allows control of the dispersion and coverage properties of the powder composition. Typically, the lower the particle size of the A and B block copolymer, the better the dispersion and coverage properties. However, if the particle size is too small, for example below 50 μm, the powder may have a greater tendency to compact, which may make it difficult to insert into the cavity. In contrast, if the particle size is too high, for example more than 500 μm, the different components of the powder would be more difficult to mix; moreover the surface covered would be less important for the same quantity of powder. Advantageously, the degradable A and B block copolymer in the form of a powder presents a particle size from 50 μm to 500 μm, from 80 μm to 500 μm, from 100 μm to 500 μm, from 120 μm to 500 μm, from 125 μm to 500 μm, from 150 μm to 500 μm, from 200 μm to 500 μm, from 250 μm to 500 μm, from 125 μm to 250 μm, from 50 μm to 450 μm, from 50 μm to 400 μm, from 50 μm to 350 μm, from 50 μm to 300 μm, from 50 μm to 250 μm, or from 50 μm to 200 μm, measured by sieving. More advantageously, the degradable A and B block copolymer in the form of a powder presents a particle size from 120 μm to 500 μm, preferably from 125 μm to 500 μm, more preferably from 125 μm to 250 μm or from 250 μm to 500 μm. Typically, the degradable A and B block copolymer in the form of a powder presents a particle size from 125 μm to 250 μm or 250 μm to 500 μm. According to the invention, the particle size of the A and B block copolymer can be measured by means well known by the one skilled in the art, for example by sieving, laser diffraction, dynamic light scattering, or image analysis methods. Typically, the particle size can be measured by sieving.

The A and B block copolymer according to the invention may be obtained via any method for synthesizing block copolymers that is known to those skilled in the art. For example, an ABA copolymer may be obtained by chain polymerization from the ends of block B. Typically, a lactide ring-opening polymerization initiated by the terminal hydroxyls of the PEO block is performed in the presence of a catalyst such as tin octanoate. This polymerization may be performed in the absence or presence of solvents. A BAB-type copolymer may be prepared, for example, by coupling methoxy-PEO onto a PLA chain whose two chain ends are carboxylic acid functions. Such a “difunctionalized” PLA is obtained, for example, by treating a PLA chain with succinic or adipic anhydride.

In the context of the invention, the powder composition advantageously comprises from 59% to 99.94%, preferably from 60% to 99.94%, preferably from 59% to 99.44%, preferably from 60% to 99.44%, preferably from 67.5% to 98.99%, of the A and B block copolymer, by weight according to the total weight of the powder composition. For example, the powder composition may comprise from 59% to 99%, or from 59% to 98%, or from 59% to 95%, or from 59% to 90%, or from 59% to 89%, or from 59% to 88%, or from 59% to 85%, or from 59% to 80%, or from 59% to 75%, or from 59% to 70%, or 99% to 99.94%, or from 98% to 99.94%, or from 95% to 99.94%, or from 90% to 99.94%, or from 89% to 99.94%, or from 88% to 99.94%, or from 85% to 99.94%, or from 80% to 99.94%, or from 75% to 99.94%, or from 70% to 99.94%, by mass of the A and B block copolymer, relative to the total mass of powder composition.

In the context of the invention, the powder composition also comprise a lubricant. Advantageously, the powder composition comprises from 0.5% to 5% of the lubricant, by mass according to the total mass of the powder composition. In particular, the powder composition can comprise from 0.5% to 5%, or from 0.5% to 4.5%, or from 0.5% to 4%, or from 0.5% to 3.5%, or from 0.5% to 3%, or from 1% to 5%, or from 1% to 4.5%, or from 1% to 4%, or from 1% to 3.5%, or from 1% to 3%, by mass of the lubricant according to the total mass of the powder composition. Advantageously, the powder composition comprises from 1% to 3% of the lubricant, by mass according to the total mass of the powder composition. For example, the powder composition may comprise 0.5%, 0.6%, 0.7%, 0.8%, 0.9%, 1%, 1.5%, 2%, 2.5%, 3%, 3.5%, 4%, 4.5%, or 5% by mass of the lubricant, relative to the total mass of powder composition. Advantageously, the lubricant is selected from the group consisting of magnesium stearate, stearic acid, sodium stearylfumarate, micronized polyoxyethyleneglycol (micronized Macrogol 6000), leucine, talc, sodium benzoate, and mixtures thereof. Preferably, the lubricant is magnesium stearate. The lubricant is preferably in the form of a powder and can be mixed with the additional components of the powder composition using any means known by the one skilled in the art. The addition of lubricant within the powder composition allows to attenuate friction and avoid the sticking with the intern surface of the inserter, and to ensure a regular flow during the injection in the uterus.

In another particular aspect of the invention, the powder composition of the invention can be free of lubricant. In this embodiment, the injection of the powder composition in the uterine cavity can be, for example, facilitated by an improved granulometry of the degradable A and B block copolymer included in the powder composition.

For example, another object of the invention can be a powder composition comprising:
a degradable A and B block copolymer, wherein:
   the A block is a polyester;
   the B block is a poly(oxyethylene) (PEO);
   the weight-average molecular mass of the blocks B is greater than or equal to 50 kDa; and
   the ethylene oxide unit/ester unit mole ratio is between 0.5 and 5;
at least one haemostatic agent; and
optionally at least one lubricant.

In particular embodiment, the powder composition can comprise:
a degradable A and B block copolymer, wherein:
   the A block is a polyester;
   the B block is a poly(oxyethylene) (PEO);
   the weight-average molecular mass of the blocks B is greater than or equal to 50 kDa; and
   the ethylene oxide unit/ester unit mole ratio is between 0.5 and 5;
at least one haemostatic agent; and
optionally at least one lubricant,
wherein the degradable A and B block copolymer is in the form of a powder with a particle size from 120 μm to 500 μm, preferably from 125 μm to 500 μm, more preferably from 125 to 250 μm or from 250 to 500 μm.

In a particular embodiment, said powder composition is free of lubricant. Accordingly, a powder composition deprived of lubricant may comprise from 60% to 99.9999% of the A and B block copolymer, by weight according to the total weight of the powder composition and from 0.0001% to 40% by mass, of haemostatic agent, in relation to the total mass of the powder composition.

In another specific embodiment, the powder composition comprises:
a degradable A and B block copolymer, wherein:
   the A block is a polyester;
   the B block is a poly(oxyethylene) (PEO);
   the weight-average molecular mass of the blocks B is greater than or equal to 50 kDa; and
   the ethylene oxide unit/ester unit mole ratio is between 0.8 and 2, preferably between 0.8 and 1.6, more preferably between more than 1 and 1.6;
at least one haemostatic agent; and
optionally at least one lubricant.

In this embodiment, the degradable A and B block copolymer is advantageously in the form of a powder with a particle size from 120 μm to 500 μm, preferably from 125 μm to 500 μm, more preferably from 125 to 250 μm or from 250 to 500 μm.

In the context of the invention, the powder composition also comprises a haemostatic agent, preferably under powder form. The haemostatic agent is an active principle that promotes coagulation and stopping the blood flow. The haemostatic agent according to the invention is advantageously selected from the group consisting of thrombin, calcium alginate, carboxymethylated starch, calcium salts such as calcium chloride, carboxymethylcellulose, gelatin, calcium ions, tranexamic acid, collagen, fibrinogen, and oxidized cellulose. Advantageously, the haemostatic agent according to the invention is selected from the group consisting of thrombin, calcium alginate, carboxymethylated starch and calcium chloride. In a preferred embodiment, the haemostatic agent is thrombin. In another preferred embodiment, the haemostatic agent is calcium alginate.

The content of the haemostatic agent in the powder composition depends on the haemostatic agent chosen. Typically, the powder composition according to the invention advantageously comprises from 0.0001% to 40% by mass, preferably from 0.0001% to 39.5%, preferably from 0.0001% to 30% by mass, of haemostatic agent, preferably under powder form, in relation to the total mass of the powder composition.

In a particular embodiment, when the haemostatic agent is thrombin, its content in the powder composition is advantageously between 0.0001% and 1%, preferably between 0.0001% and 0.1%, preferably between 0.001% and 0.1%, more particularly between 0.005% and 0.05%.

In another particular embodiment, when the haemostatic agent is calcium alginate or carboxymethylated starch, its content in the powder composition is advantageously between 5% and 40%, preferably between 8% and 40%, preferably between 5% and 39.5%, preferably between 8% and 39.5% preferably between 8% to 32%, or between 20% and 30%, or between 25% and 30%, by mass relative to the total mass of the powder composition.

In another particular embodiment, when the haemostatic agent is calcium chloride, its content in the powder composition is advantageously between 0.01% and 5%, preferably between 0.1% and 5%, preferably between 0.5% and 5%, preferably between 1% and 5%, preferably between 1% and 4%, preferably between 1% and 3%, preferably 2%, by mass relative to the total mass of the powder composition.

In a particular embodiment, the powder composition according to the invention may also comprise a carrier, for the haemostatic agent, also called filler. Advantageously, the powder composition comprises from 0% to 40% of the carrier, by mass according to the total mass of the powder composition. In particular, the powder composition can comprise from 0% to 40%, or from 0% to 39.5%, or from 1% to 40%, or from 5% to 40%, or from 5% to 30%, or from 5% to 20%, or from 7% to 19%, or from 8% to 40%, from 10% to 40%, or from 10% to 35%, by mass of the carrier according to the total mass of the powder composition. Advantageously, the powder composition comprises from 5% to 30% of the carrier, by mass according to the total mass of the powder composition. For example, the powder composition may comprise 0%, 5%, 7%, 8%, 10%, 15%, 19%, 20%, 25%, 30%, 35%, 40%, by mass of the carrier, relative to the total mass of powder composition. Advantageously, the carrier is selected from the group consisting of lactose, carboxymethylated starch, polyethylene glycol (PEG), poly(oxyethylene) (PEO), and carboxymethylcellulose. Preferably, the carrier is lactose or carboxymethylated starch. The addition of a carrier for the haemostatic agent within the powder composition makes it possible to improve the dispersion and homogeneity of the powder composition as well as the dispersion of the haemostatic agent, and thus to accelerate the contact of the powder composition according to the invention with the walls of the uterine cavity. The mixing of the haemostatic agent with the carrier may be performed by any means known to those skilled in the art, for example by dissolution of the haemostatic agent in a carrier solution followed by drying or lyophilisation.

In a particular embodiment, the powder composition according to the invention comprises:
From 59% to 99.44% of the degradable A and B block copolymer;
From 0.5% to 5% of the lubricant,
From 0.0001% and 1% of thrombin, and
From 0% to 39.5% of the carrier,
   by mass relative to the total mass of the powder composition.

In a particular embodiment, the powder composition according to the invention comprises:
From 94.9999% to 99.44% of the degradable A and B block copolymer;
From 0.5% to 5% of the lubricant,
From 0.0001% and 1% of thrombin,
   by mass relative to the total mass of the powder composition.

In a particular embodiment, the powder composition according to the invention comprises:
From 80% to 90% of the degradable A and B block copolymer;
From 1% to 3% of the lubricant,
From 0.0001% and 0.1% of thrombin, and
From 7% to 19% of the carrier,
   by mass relative to the total mass of the powder composition.

In a particular embodiment, the powder composition according to the invention comprises:
From 80% to 90% of the degradable A and B block copolymer;
From 1% to 3% of the lubricant,
From 0.5% to 3% of calcium chloride, and
From 7% to 19% of the carrier,
   by mass relative to the total mass of the powder composition.

In a particular embodiment, the powder composition according to the invention comprises:
From 67% to 89% of the degradable A and B block copolymer;
From 1% to 3% of the lubricant,
From 8% to 32% of calcium alginate,
   by mass relative to the total mass of the powder composition.

In a particular embodiment, the powder composition according to the invention comprises:
From 67% to 89% of the degradable A and B block copolymer;
From 1% to 3% of the lubricant,
From 8% to 32% of carboxymethylated starch,
   by mass relative to the total mass of the powder composition.

In another particular aspect, the powder composition according to the invention comprises:
From 59% to 99.44% of the degradable A and B block copolymer in the form of a powder with
a particle size from 250 μm to 500 μm;
From 0% to 5% of lubricant,
From 0.0001% and 1% of thrombin, and
From 0% to 39.5% of the carrier,
   by mass relative to the total mass of the powder composition.

In a particular embodiment, the powder composition according to the invention comprises:
From 94.9999% to 99.44% of the degradable A and B block copolymer in the form of a powder with a particle size from 250 μm to 500 μm;
From 0% to 5% of lubricant,
From 0.0001% and 1% of thrombin,
   by mass relative to the total mass of the powder composition.

In a particular embodiment, the powder composition according to the invention comprises:
From 80% to 90% of the degradable A and B block copolymer in the form of a powder with a particle size from 250 μm to 500 μm;
From 0% to 3% of lubricant,
From 0.0001% and 0.1% of thrombin, and
From 7% to 19% of the carrier,
   by mass relative to the total mass of the powder composition.

In a particular embodiment, the powder composition according to the invention comprises:
From 80% to 90% of the degradable A and B block copolymer in the form of a powder with a particle size from 250 μm to 500 μm;
From 0% to 3% of lubricant,
From 0.5% to 3% of calcium chloride, and
From 7% to 19% of the carrier,
   by mass relative to the total mass of the powder composition.

In a particular embodiment, the powder composition according to the invention comprises:
From 67% to 89% of the degradable A and B block copolymer in the form of a powder with a particle size from 250 μm to 500 μm;
From 0% to 3% of lubricant,
From 8% to 32% of calcium alginate,
   by mass relative to the total mass of the powder composition.

In a particular embodiment, the powder composition according to the invention comprises:
From 67% to 89% of the degradable A and B block copolymer in the form of a powder with a particle size from 250 μm to 500 μm;
From 0% to 3% of lubricant,
From 8% to 32% of carboxymethylated starch,
   by mass relative to the total mass of the powder composition.

Particular examples of the powder composition of the invention can be:
97.97% of the ABA triblock copolymers+0.03% Thrombin+2% magnesium stearate; or
68.5% of the ABA triblock copolymers+0.03% Thrombin+29.97% carboxymethylated starch+1.5% magnesium stearate; or
68% of the ABA triblock copolymers+30% carboxymethylated starch+2% magnesium stearate; or
68.25% of the ABA triblock copolymers+29.25% calcium alginate+2.5% magnesium stearate; or
95.5% of the ABA triblock copolymers+2% CaCl2+2.5% magnesium stearate; or
79% of the ABA triblock copolymers+18% Lactose+2% CaCl2+1% magnesium stearate; or
85% of the ABA triblock copolymers in the form of a powder with a particle size from 250 μm to 500 μm+15% calcium alginate.

In a particular embodiment, the powder composition according to the invention comprises only the a degradable A and B block copolymer, the lubricant, the haemostatic agent and optionally the carrier of the haemostatic agent. In a particular embodiment, the powder composition according to the invention consists of a degradable A and B block copolymer, the lubricant, the haemostatic agent and optionally the carrier of the haemostatic agent. In another particular embodiment, the powder composition according to the invention consists of a degradable A and B block copolymer in the form of a powder with a particle size from 250 μm to 500 μm, the haemostatic agent and optionally the carrier of the haemostatic agent.

In another particular embodiment, the powder composition according to the invention may also include an additional additive or active principle, such as a therapeutic molecule such as an antibiotic or a vasoconstrictor. This additive or active principle may, for example, be added to the composition in the form of powder, so as to be dispersed in the powder composition. Preferentially, the additional active principle is capable of diffusing outwards from the material when it is in an aqueous or humid medium. By way of example, the additional active principle could be a vasoconstrictor.

The preparation of the powder composition according to the invention may be performed by any means known to those skilled in the art, and notably by mixing together each ingredient in powder forms. Other methods for preparing the powder composition of the invention could be for example spray drying, granulation preferably wet granulation, physical mixing, or solubilisation/precipitation in a non-solvent with stirring.

In the context of the invention, the powder composition according to the invention advantageously allows a release of at least 30% of the haemostatic agent initially present within 10 minutes or less after introduction of the powder composition of the invention into a body cavity, preferably into the uterine cavity. In particular, the powder composition according to the invention advantageously allows a release into a body cavity, in particular into the uterine cavity, of at least 30% of the haemostatic agent initially present within less than 8 minutes, 6 minutes, 5 minutes, 4 minutes, 3 minutes, 2 minutes, or 1 minute (limits included), after introduction of the powder composition of the invention into a body cavity, preferably into the uterine cavity.

Advantageously, the powder composition according to the invention allows a release into a body cavity, in particular into the uterine cavity, of at least 50%, preferably at least 60%, of the haemostatic agent initially present within 10 minutes or less after introduction of the powder composition of the invention into a body cavity, preferably into the uterine cavity. In particular, the powder composition according to the invention advantageously allows a release into a body cavity, in particular into the uterine cavity, of at least 50%, preferably at least 60%, of the haemostatic agent initially present within less than 8 minutes, 6 minutes, 5 minutes, 4 minutes, 3 minutes (limits included), after introduction of the powder composition of the invention into a body cavity, preferably into the uterine cavity.

Advantageously, the powder composition according to the invention allows a release into a body cavity, in particular into the uterine cavity, of at least 80%, preferably at least 90%, of the haemostatic agent initially present within 10 minutes or less after introduction of the powder composition of the invention into a body cavity, preferably into the uterine cavity. In particular, the powder composition according to the invention advantageously allows a release into a body cavity, in particular into the uterine cavity, of at least 80%, preferably at least 90%, of the haemostatic agent initially present within less than 10 minutes, 9 minutes or 8 minutes, after introduction of the powder composition of the invention into a body cavity, preferably into the uterine cavity.

In a preferred embodiment, the powder composition according to the invention advantageously allows a release into a body cavity, in particular the uterine cavity of:
at least 30% of the haemostatic agent initially present within 8 minutes or less;
at least 50% of the haemostatic agent initially present within 10 minutes or less; and/or
at least 80% of the haemostatic agent initially present within 30 minutes or less;
   after introduction of the powder composition of the invention into a body cavity, preferably into the uterine cavity.

In a preferred embodiment, the powder composition according to the invention advantageously allows a release into a boy cavity, in particular into the uterine cavity, of:
at least 50% of the haemostatic agent initially present within 6 minutes or less;
at least 60% of the haemostatic agent initially present within 10 minutes or less; and/or
at least 80% of the haemostatic agent initially present within 30 minutes or less;
   after introduction of the powder composition of the invention into a body cavity, preferably into the uterine cavity.

In a preferred embodiment, the powder composition according to the invention advantageously allows a release into a boy cavity, in particular into the uterine cavity, of:
at least 30% of the haemostatic agent initially present within 1 minutes or less;
at least 60% of the haemostatic agent initially present within 5 minutes or less; and/or
at least 80% of the haemostatic agent initially present within 10 minutes or less;
   after introduction of the powder composition of the invention into a body cavity, preferably into the uterine cavity.

In the context of the invention, the release profile of the haemostatic agent into the body cavity, in particular the uterine cavity, is measured according to the ELISA (Enzyme-Linked Immunosorbent Assay) method.

In the context of the invention, the rapid release of the haemostatic agent onto the body cavity wall, in particular onto the uterine wall, enables fast and efficient treatment of haemorrhage, in particular uterine haemorrhage.

An additional particularly advantageous feature of the powder composition according to the invention administered in the uterine cavity is that it has anti-adhesion properties. Once haemostasis has been achieved, the cicatrization process follows its course and there is a risk that this may lead to the two walls of the uterus coming together in the form of a fibrous bridge known as an adhesion or synechia. The presence of the powder composition with anti-adhesion properties makes it possible to create a physical and mechanical barrier between the walls, and thus allows cicatrization without synechiae.

An additional particularly advantageous feature of the powder composition according to the invention is that it is degradable in an aqueous or humid medium. In particular, the powder composition according to the invention degrades after a residence time in the body cavity, in particular the uterine cavity, of between 2H and 30 days, preferentially between 12H and 20 days, and more preferentially between 1 and 15 days. The degradation of the powder composition is due to the progressive hydrolysis of the ester bonds of the polyester blocks followed by dissolution of the PEO-containing blocks. The loss of mechanical properties of the material is directly related to its degradation. The degradation may be evaluated by measuring the decrease in molecular weight of a strip of material over time, after immersion at 37° C. in saline medium (PBS 1×) with stirring, for example by Size Exclusion Chromatography. It is also possible to evaluate the decrease in the dynamic viscosity of the material. The degradation properties of the powder composition according to the invention thus allow the powder composition to remain intact in the body cavity, in particular the uterine cavity for a time that is sufficient to treat haemorrhage, in particular uterine haemorrhage, and then to degrade sufficiently to enable it to be eliminated naturally.

In the context of the invention, the powder composition can be considered as a degradable system for the release of a haemostatic agent into a body cavity. In particular, the powder composition can be considered as a degradable intrauterine system for the release of a haemostatic agent into the uterine cavity.

### Uses of the Powder Composition of the Invention

Another object of the invention is the powder composition as defined above, for the administration of the haemostatic agent into a body cavity, preferably into the uterine cavity.

Another object of the invention is the powder composition as defined above, for use in a method of preventing and/or treating haemorrhage, preferably uterine haemorrhage.

Another object of the invention is the powder composition as defined above, for use in a method of preventing and/or treating haemorrhage, wherein the composition is for an administration in a body cavity of a subject.

Another object of the invention is the powder composition as defined above, for use in a method of preventing and/or treating uterine haemorrhage, wherein the composition is for an administration in the uterine cavity of a subject.

Another object of the invention is a method for preventing and/or treating haemorrhage in a subject, comprising a step of administering the powder composition as defined above into the body cavity to be treated, in a subject in need thereof.

Another object of the invention is a method for preventing and/or treating uterine haemorrhage in a subject, comprising a step of administering the powder composition as defined above into the uterine cavity, in a subject in need thereof.

Another object of the invention is the use of the powder composition as defined above for the manufacture of medicaments for use in a method of preventing and/or treating haemorrhage preferably uterine haemorrhage. In particular, the composition is for an administration into a body cavity of a subject, in particular into the uterine cavity.

In the context of the invention, haemorrhage is an acute loss of blood from a damaged blood vessel. The haemorrhage can occur in any part of the body, in particular the mammal body, preferably the human body. Common sources of haemorrhage include organ damage (hepatic, splenic, renal, adrenal), vascular injury, gynecologic/obstetric procedure complications, or coagulopathies. For example, the haemorrhage can be selected in the group consisting of uterine haemorrhage, hemothorax, abdominal haemorrhage, brain haemorrhage, organ haemorrhage (such as hepatic, splenic, renal, adrenal), oral haemorrhage, nasal haemorrhage, rectal haemorrhage, vaginal haemorrhage, preferably uterine haemorrhage.

### The Kit

Another object of the invention is a kit comprising (i) the powder composition of the invention as defined above, preferably in a content from 500 mg to 2000 mg; and (ii) means for inserting said powder composition into a body cavity. The kit according to the invention advantageously comprises means for inserting and placing the material into a body cavity.

Another object of the invention is a kit comprising (i) the powder composition of the invention as defined above, preferably in a content from 500 mg to 1000 mg; and (ii) means for inserting said powder composition into a body cavity, preferably into the uterine cavity. The kit according to the invention advantageously comprises means for inserting and placing the material into a body cavity, in particular the uterine cavity.

The kit according to the invention advantageously comprises a pharmaceutically effective dose of the powder of the invention. In a specific embodiment, the kit comprises a single dose of the powder of the invention. In another specific embodiment, the kit can comprise at least two doses of the powder of the invention.

For example, the kit according to the invention may comprise a hollow cylindrical inserter in the tube of which the powder composition is housed. Advantageously, in order to minimize the dimensions of the inserter, the powder composition is housed in the tube in a compacted form. By “compacted form” it is meant that the powder is held in a defined volume.

The kit advantageously includes a plunger mounted to slide in translation at a distal end of the inserter, the opposite proximal end being the end through which the inserter is intended to be introduced into the body cavity, in particular the uterine cavity. The plunger comprises or consists of a rod which, when pushed inside the bore of the inserter, towards the proximal end, drives the powder composition in translation towards the outside of the inserter.

Advantageously, the plunger comprises stop means at the proximal end of the extrusion, said stop means being intended to come up against the wall of the bore bordering the proximal end of the inserter, so as to inform the person handling the kit that the powder composition has been completely ejected from the inserter and is in position into the body cavity, in particular the uterine cavity. It then suffices to remove the insertion means/inserter assembly by simply pulling outwards.

Such a kit allows the powder composition according to the invention to be reliably introduced and homogeneously dispersed in the body cavity, in particular the uterine cavity.

The kit according to the invention may notably be used in the case of patients suffering from uterine haemorrhage. The compacted form of the material and the use of a small applicator facilitate its insertion in the often sensitive uterine cavity of these patients. In addition, its natural elimination during the menstrual cycle avoids the need for an additional intervention on the patient by medical staff to remove said device.

The invention will now be illustrated with the aid of the examples below. These examples are merely presented as non-limiting indications of the invention.

### EXAMPLES

### Example 1: Preparation of a Powder Composition According to the Invention

### 1. Synthesis of ABA Triblock Copolymers

a. Materials

Commercial poly(ethylene oxide) (PEO): Supplier Sigma-Aldrich, CAS No. 25322-68-3. Commercial PEO was analysed in the laboratory by Size Exclusion Chromatography (SEC) so as to determine its weight average molar mass (Mw). The analysis was performed in an analytical solvent (dimethylformamide), and the Mw was determined using a poly(ethylene glycol) calibration range. The weight average molar mass Mw is 95 000 Da and its inherent viscosity is 0.16 ml/mg. Commercial D,L-lactide: supplier Corbion Purac, CAS No. 95-96-5.

b. Method

The ABA triblock is synthesized in the following manner:

PEO (Mw 95 000) (200 g) and D,L-lactide (458 g) are dried under vacuum at room temperature for 24 hours. PEO and D,L-lactide are introduced into a polymerization flask in the presence of tin octanoate (85 mg). 10 successive cycles of vacuum (10-3 bar) and inertization with argon are then performed. The mixture is then heated to 140° C. and a further 10 successive vacuum and argon-inertization cycles are performed. The mixture is returned to room temperature and then placed in an ice bath. Once crystallized, the reaction mixture is placed under dynamic vacuum for 30 min and then sealed under dynamic vacuum. The mixture is then placed in an oven with mechanical rotation at 140° C. for 3 days. The mixture is dissolved in dichloromethane and precipitated from an ether/ethanol mixture. The precipitate is recovered and then dried under vacuum for 24 hours.

c. Characterization

The final composition of the copolymer was determined by ¹H NMR proton nuclear magnetic resonance and an EO/LA mole ratio of 1 is deduced. The molar ratio between ethylene oxide units (EO) and lactic acid units (La) has been determined as followed: EO/LA=[peak area of EO unit methylenes (3.5 ppm)/number of proton in EO unit]/[peak area of LA unit methine (5.1 ppm)/number of proton in LA unit].

Two-dimensional NMR analysis (DOSY) indicates that the synthesis did indeed result in an ABA triblock (PLA50-PEO-PLA50).

The copolymer was also analysed by Size Exclusion Chromatography (SEC) so as to determine its average molar mass Mw and its dispersity Ip.

With an analytical solvent such as dimethylformamide and using a poly(ethylene glycol) calibration range, an Mw of 123 000 Da and a dispersity in the region of 5 were obtained.

In addition, Thermogravimetric Analysis (TGA) made it possible to determine the degradation temperature of the copolymer, which is 256° C.

2. Preparation of the powder composition according to the invention

a. Materials
Thrombin of human origin, in lyophilized form, supplier Sigma Aldrich (CAS #9002-04-4). The activity of the protein, included in the certificate of analysis of the product, is: >400 NIH units/mg protein.
The triblock ABA powder as prepared above (section 1) is used in the composition of powders A1 to A4, B1 to B5, C1 to C5, D1 to D3 and E1 to E2.
A triblock ABA powder as prepared above (section 1) but with a EO/LA mole ratio of 1.56 is used in the composition of powders G0 to G6.
PEO, with an average molecular weight Mw of 100 kDA, supplied by Sigma Aldrich (CAS #25322-68-3)—Average granulometry around 150 μm.
Calcium Alginate, Alginic acid sodium salt from brown algae, Algin, Sodium alginate, supplied by Sigma Aldrich (CAS #9005-38-3)—Particles size D_{98%}<160 μm.
CaCl₂), anhydrous, granular, ≤7.0 mm, ≥93.0%, supplied by Sigma Aldrich (CAS #10043-52-4).
Lactose, white crystalline powder, supplied by ArmorPharma (reference EXCIPRESS SD2L).
Carboxymethylated starch, sodium starch glycolate supplied by Roquette (reference GLYCOLYS)
Particles size D₉₈%<105 μm.
Magnesium Stearate, Stearic acid magnesium salt, supplied by Sigma Aldrich (CAS #557-04-0).
Sodium Stearyl Fumarate, (E)-2-Butenedioic acid monooctadecyl ester sodium salt, supplied by Sigma Aldrich (CAS #4070-80-8).
Talc, Hydrated Magnesium Silicate powder, supplied by Cooper.
   b. Powder Preparation

### Powder Containing a Carrier and Thrombin

The carrier is solubilized in a saline solution (pH between 6.5 and 7.5) under agitation at room temperature. Lyophilized thrombin is then solubilized in this solution. The resulting Carrier-Thrombin solution is placed in a freeze-dryer at −52° C. under 0.06 mbar for 16 hours.

The lyophilized Carrier/Thrombin mix is then mixed together with Triblock ABA powder (granulometry included between 125 μm and 500 μm) and lubricant using a three-dimensional mixer. The ingredients and contents are disclosed in Table 1 below.

Powder Containing a Haemostatic Agent without Carrier

Triblock ABA powder (granulometry included between 125 μm and 500 μm), haemostatic agent and lubricant are mixed using a three-dimensional mixer. The ingredients and contents are disclosed in Table 1 below.

TABLE 1

| | Triblock | | | | | | |
|---|---|---|---|---|---|---|---|
| | ABA | Haemostatic agent | | Lubricant | | Carrier | |
| | copolymers | | Content | | Content | | Content |
| Prototypes | % (w/w) | Name | % (w/w) | Name | % (w/w) | Name | % (w/w) |
| A1 | 90 | Thrombin | 0.007 | — | — | PEO | 9.993 |
| A2 | 90 | Thrombin | 0.013 | — | — | PEO | 9.987 |
| A3 | 90 | Thrombin | 0.026 | — | — | PEO | 9.974 |
| A4 | 90 | — | — | — | — | PEO | 10 |
| Comparative | | | | | | | |
| B1 | 88 | Thrombin | 0.05 | Magnesium | 2 | Lactose | 9.95 |
| | | | | stearate | | | |
| B2 | 88 | Thrombin | 0.02 | Magnesium | 2 | Lactose | 9.98 |
| | | | | stearate | | | |
| B3 | 88 | — | — | Magnesium | 2 | Lactose | 10 |
| | | | | stearate | | | |
| B4 | 88 | Thrombin | 0.02 | Magnesium | 2 | Carboxy- | 9.98 |
| | | | | stearate | | methylated | |
| | | | | | | starch | |
| B5 | 88 | — | — | Magnesium | 2 | Carboxy- | 10 |
| | | | | stearate | | methylated | |
| | | | | | | starch | |
| C1 | 68.25 | Calcium | 29.25 | Magnesium | 2.5 | — | — |
| | | alginate | | stearate | | | |
| C2 | 95.5 | CaCl₂ | 2 | Magnesium | 2.5 | — | — |
| | | | | stearate | | | |
| C3 | 68.25 | Carboxy- | 29.25 | Magnesium | 2.5 | — | — |
| | | methylated | | stearate | | | |
| | | starch | | | | | |
| C4 | 97.485 | Thrombin | 0.015 | Magnesium | 2.5 | — | — |
| | | | | stearate | | | |
| C5 | 97.47 | Thrombin | 0.03 | Magnesium | 2.5 | — | — |
| | | | | stearate | | | |
| D1 | 96 | Thrombin | 0.01 | — | — | PEO | 3.99 |
| D2 | 97 | Thrombin | 0.01 | — | — | PEO | 2.99 |
| D3 | 97 | — | — | — | — | PEO | 3 |
| comparative | | | | | | | |
| E1 | 98 | CaCl₂ | 2 | — | — | — | — |
| E2 | 70 | Calcium | 30 | — | — | — | — |
| | | alginate | | | | | |
| G0 | 85% | Calcium | 15% | — | — | — | — |
| | | alginate | | | | | |
| G1 | 84.15% | Calcium | 14.85% | Magnesium | 1% | — | — |
| | | alginate | | stearate | | | |
| G2 | 84.15% | Calcium | 14.85% | Sodium | 1% | — | — |
| | | alginate | | Stearyl | | | |
| | | | | Fumarate | | | |
| G3 | 84.15% | Calcium | 14.85% | Talc | 1% | — | — |
| | | alginate | | | | | |
| G4 | 82.45% | Calcium | 14.55% | Magnesium | 3% | — | — |
| | | alginate | | stearate | | | |
| G5 | 82.45% | Calcium | 14.55% | Sodium | 3% | — | — |
| | | alginate | | Steary1 | | | |
| | | | | Fumarate | | | |
| G6 | 82.45% | Calcium | 14.55% | Talc | 3% | — | — |
| | | alginate | | | | | |

### Example 2: Evaluation of the Swelling Properties of Powder Triblock ABA

At T=0, 500 mg of triblock ABA as prepared in example 1 have been placed in a crystallizer, and then 30 mL of water have been added. The powder was left in the water for 10 minutes. Absorbent paper was used to absorb the water and then the powder was recovered. The powder has been weighted: 1.49 g at min. The powder has almost tripled its initial mass (mass increase by 200%).

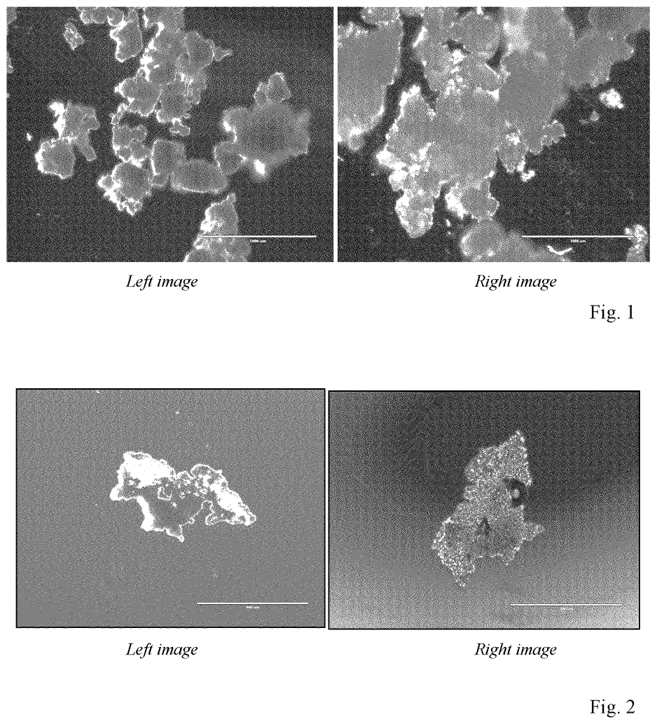

FIG. 1 shows that the powder has swollen and the grains have agglomerated at T=10 min, compared with the powder at T=0 min.

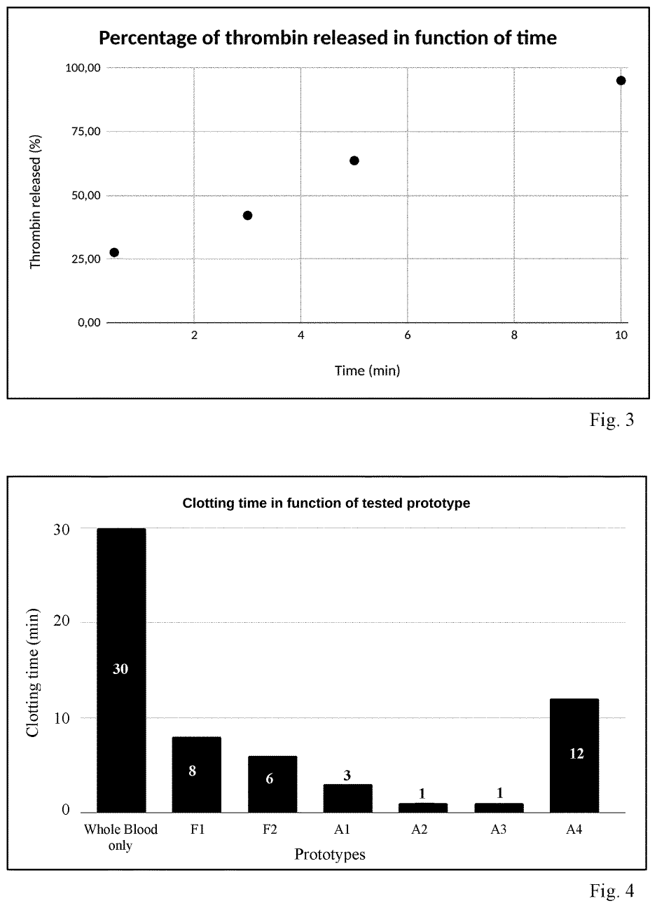

One grain of Triblock ABA has been placed under the microscope; a photo has been taken at T=0; then 10 μL of water has been dropped on the grain. After 30 seconds, a photo has been taken in order to observe whether the grain had absorbed water (FIG. 2).

The powder surface has been measured using the software ImageJ (based on the scale of the images), in order to evaluate the surface increase between T=0 and T=30 seconds. The results are the following:
Surface at T=0:71963 μm² (0.072 mm²)
Surface at T=30 sec: 91965 μm² (0.092 mm²)

We obtain an increase of 20 mm² in 30 seconds; that means that the powder has absorbed water.

### Example 3: Evaluation of the Thrombin Release Kinetics

### In-Vitro Release Method

The powder is placed in a 50 ml vial containing 10 ml of phosphate buffer (pH 7.4). The vial is placed at 37° C. under mechanical agitation (87 rpm). 3 samples of luL are collected after introduction at 30 sec, 3 min, 5 min and 10 min release. Each sample is analyzed using the enzyme-linked immunosorbent assay (ELISA). The amount of thrombin released is calculated for each sampling time from the equation of the the equation of the calibration curve obtained with the standard range.

### Method for the Determination of Thrombin

The ELISA (supplied by Abcam; reference ab270210) used is designed for the quantitative measurement of thrombin. The thrombin (analyte) present in the solution collected during the release assay, is captured by a capture antibody, and by a detector antibody conjugated to a reporter, which will reveal the amount of thrombin present in the analyzed sample. The whole complex (capture antibody capture antibody/analyte/detector antibody) is then immobilized by immunoaffinity of an anti-labeling antibody covering the well. A signal is thus generated, proportional to the quantity of analyte (thrombin) bound to the antibody complex. The intensity of the signal is measured at 450 nm using a microplate reader.

### Calculation Method

A series of dilutions is performed from a stock solution of thrombin (1689600 μg/mL), to obtain a concentration range from 0 to 8500 μg/mL. The optical density (OD) measurements at 450 nm, obtained for each concentration, allow to draw a standard curve that relates the optical density to the thrombin concentration. The equation of this curve will determine the thrombin concentration of unknown samples. Then, based on the amount of thrombin contained in the powder, we can evaluate the percentage of thrombin released as a function of time.

### Powder Composition

The powder used is the powder prototype A1 described in Example 1.

### Results

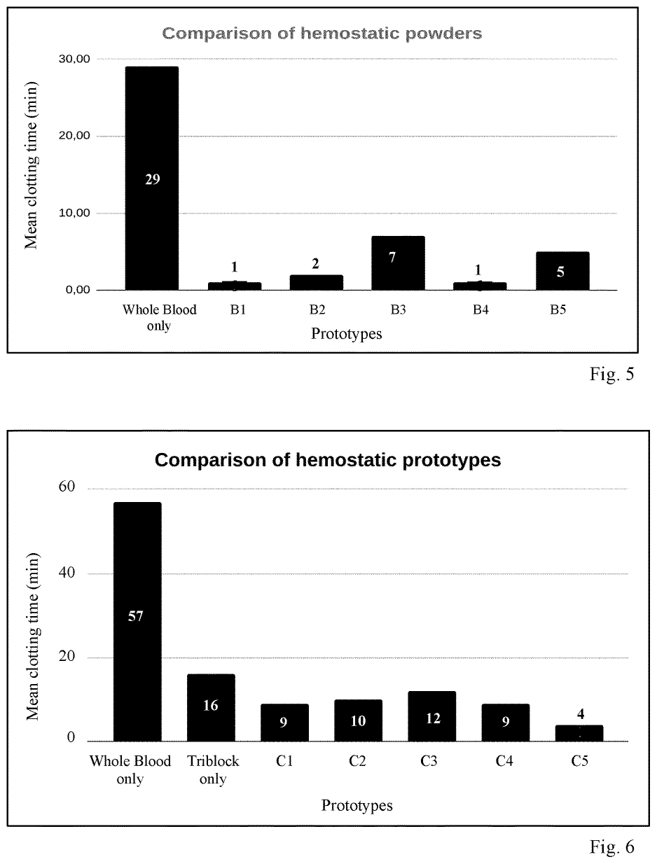

The calibration curve is linear (R2=0.98) over the concentration range of 0 to 8500 μg/mL. The same calibration curve was used to determine the concentration of thrombin in solution, released by the powder A1. The percentage of thrombin released over time from A1 is shown in FIG. 3 and Table 2. Under in-vitro release conditions, thrombin is released from the powder over time. The release of thrombin begins at about 30 seconds and at least 95% of the thrombin contained in the powder is released after 10 minutes.

TABLE 2

| Time (min) | Percentage of thrombin released (%) |
|---|---|
| 0.5 | 27.58 |
| 3 | 42.10 |
| 5 | 63.60 |
| 10 | 95.04 |

### Example 4: Evaluation of the Haemostatic Properties (In Vitro)

a. Material and Method

### Hemostasis Support

Whole blood with CPD (citrate phosphate dextrose) is provided by the Etablissement Français du Sang. Before being used for coagulation tests, this blood must be recalcified; for this purpose Calcium Chloride (CaCl₂—CAS 10043-52-4-supplier Sigma-Aldrich) is added to the blood. For subsequent tests, 3 mL of CaCl₂ (concentration 0.122 mol/L) is added to 30 mL of whole blood.

### In-Vitro Model

30 mL of whole blood with CPD (not recalcified), contained in a 50 mL falcon, is heated in a water bath at 37° C. for 30 minutes before the start of the assay. After addition of CaCl₂, 10 mL of the recalcified blood is introduced into a plastic uterine cavity model (dimensions: height 75 mm; large base 45 mm; small base 12 mm). This model is held upside down (small base/open part up) to avoid blood loss during the test. The powder prototype to be tested is then inserted into the model using an inserter. The whole assembly is placed in an oven at 37° C.

### Determination of Clotting Time

Coagulation is observed visually. The clotting time is the time when all the blood in the uterine cavity model has clot. To determine this time, the model (containing the blood and the prototype) is rotated 180° every minute for the first 10 minutes, then every 2 minutes until coagulation is observed. The tests are performed in triplicate; then the average clotting time obtained is calculated.

### Prototypes Tested

The powder prototypes A1-A4, B1-B5 and C1-C5 used for the in-vitro tests have been prepared using the method described in Example 1. The powder composition is detailed in Table 1 included in Example 1.

The comparative film prototypes F1 and F2 recited hereinbelow have been obtained by preparing a triblock ABA powder as described in Example 1 and then by forming a film by hot pressing: 2.5 mg of powder are pressed between two plates heated to 85° C., for 9 minutes and under a pressure of 20 MPa. The film obtained, 500 μm thick, is then cut with a sample punch to obtain films having the following dimensions: Height 25 mm; Large base 20 mm; Small base 10 mm.
F1: Uterine film impregnated with THR 100 NIH Units. Composition: 99,984% Triblock ABA and 0.016% pure Thrombin.
F2: THR coated uterine film 100 NIH Units. Composition: 80% Triblock ABA, 19,987% PEO and 0.013% pure Thrombin.
   b. Results
   b1—Comparison of Powders Prototypes A1 to A4 with Uterine Films F1 and F2

FIG. 4 presents the clotting time (min) as a function of the prototypes tested.

The following observations can be made:
The blood clotting time is shorter when the blood is in contact with the prototypes tested, in comparison with whole blood only.
The more thrombin units there are, the faster is the clotting time (A1-100 Units vs A2-200 Units vs A3-400 Units).
Clotting time of powder mix that includes thrombin (A1, A2 & A3) is better than a powder mix without thrombin (A4).
For the same number of thrombin NIH Units, the clotting time of the powder mix (A1) is better than the clotting time of the films (F1 & F2). We can assume that the blood is partially absorbed by the powder and the thrombin is immediately available from the powder, unlike film forming, where blood is absorbed more slowly and thrombin takes longer to be released.
   b2—Comparison of Prototypes B to B5

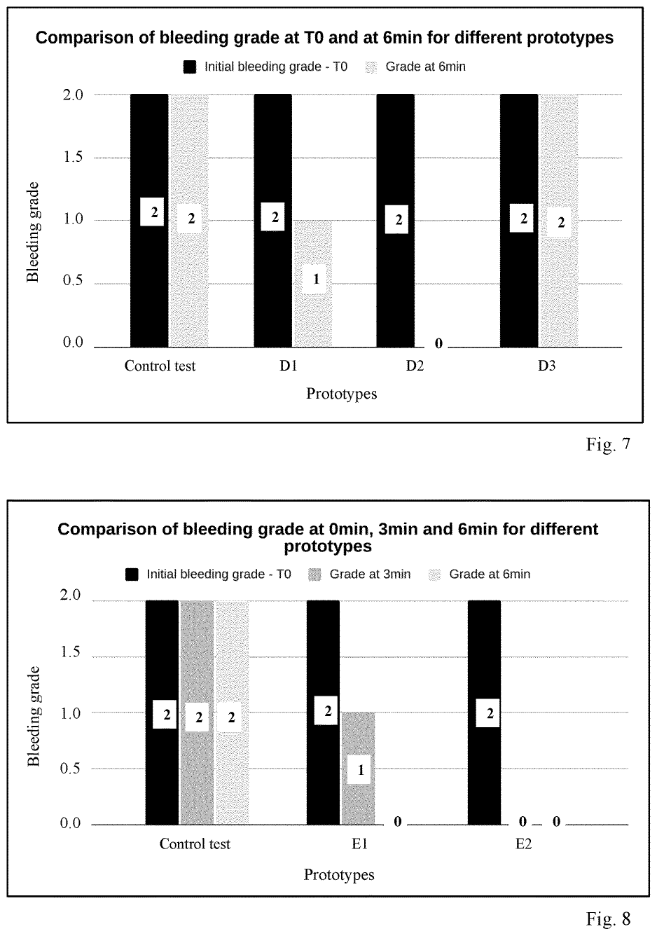

FIG. 5 presents the clotting time (min) as a function of the prototypes tested. Prototypes including lactose or carboxymethylated starch as carriers are tested.

The following observations can be made:
It can be observed that the blood clotting time is shorter when the blood is in contact with the prototypes tested, in comparison with whole blood only.
Clotting time of powder mix that includes thrombin (B1, B2, B4) is better than a powder mix without thrombin (B3, B5).
The more thrombin units there are, the faster is the clotting time (B1-100 Units vs B2-50 Units).
The clotting time of Carboxymethylated starch mix with triblock (B5, without thrombin) is better than the clotting time of Lactose mix with triblock (B3, without thrombin). No difference can be seen between these same prototypes including thrombin (B1, B4), when Lactose and Carboxymethylated starch are used as haemostatic carriers.

### B3—Comparison of Prototypes C1 to C5

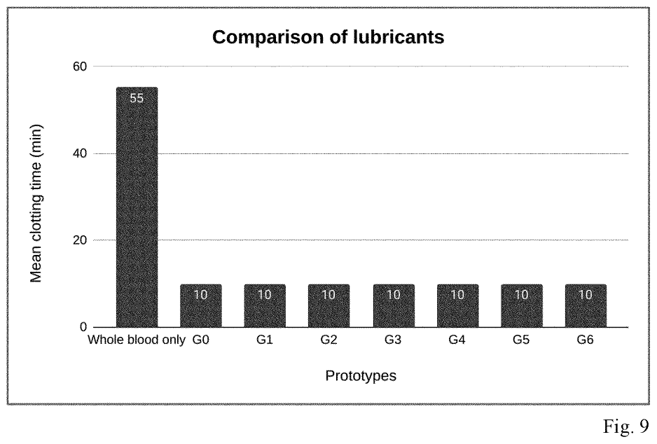

FIG. 6 presents the clotting time (min) as a function of the prototypes tested. Prototypes include different hemostatic agents mixed with triblock and magnesium stearate.

The following observations can be made:
It can be observed that the blood clotting time is shorter when the blood is in contact with the prototypes tested, in comparison with whole blood only.
The powder mix that includes 100 NIH Units of thrombin (C5) is better than the other powder mix.
The clotting time of triblock mixed with any haemostatic agent is better than the triblock only.
Prototypes containing calcium (C1, C2) have a better clotting time than the prototype containing carboxymethylated starch (C3).
The clotting time of the prototype including calcium alginate (C1) is equivalent to the prototype containing 50 NIH Units of thrombin (C4).

### Example 5: Evaluation of the Haemostatic Properties (In Vivo)

i. Bleeding Scale

A standardised semi-quantitative bleeding scale is used to evaluate bleeding before in order to define the initial bleeding severity that should reflect the clinical use. This bleeding scale will be used throughout the test too to observe the evolution of the bleeding. See below the scale:
0=No bleeding
0.5=Ooze (blood observed at the edges, but not flowing)
1=Very slight bleeding (blood flows very slowly from the site)
2=Slight (blood flows slowly)
3=Moderate (blood flowing fast without pulsatile action)
4=Severe (blood flowing fast, pulsing and squirting out of wound)

A bleeding grade from 1 to 2 is preferred for this test.

ii. In-Vivo Model

The pig is selected due to its anatomical size and organ structure, which are similar to that of humans as well as similarity to humans with respect to the coagulation system.

A section is made on the liver of the pig allowing the insertion of a prototype inside parenchyma. The haemostatic prototype will be in direct contact with the bleeding wound.

To obtain a grade of 1 to 2, the following section dimensions are used: 1 cm length; 0.5 cm in depth.

iii. Determination of Haemostatic Properties

The evolution of the bleeding grade is observed in order to evaluate the haemostatic performance of the prototypes (i.e. ability to decrease the bleeding grade).

The initial bleeding is scored approximately 1 min after section creation, when the severity of the bleed is stable. The prototype is then inserted into the wound using a syringe. The timer is triggered once the prototype is in place. Haemostasis performance (bleed score) is scored at 3 minimum time points (e.g. 1, 3 and 6 minutes) using the bleeding scale defined above. The test is stopped at 6 minutes.

iv. Prototypes Preparation

The powder prototypes D1-D3 and E1-E2 have been prepared with the method described in Example 1 and the quantities are the ones detailed in Table 1. The control test corresponds to: no prototype inserted into the wound.

v. Results
a. Comparison of Prototypes D1, D2 and D3

The results are summarized in FIG. 7. A comparison between the initial bleeding grade and the bleeding grade at 6 minutes is performed to evaluate the haemostatic performance of the prototypes tested.

The following observations can be made:
At 6 minutes, the blood flow remains the same when there is no thrombin (Control Test, D3).
The blood flow decreases by half (D1), or the bleeding stops completely (D2) when a thrombin powder mix is introduced into the wound.
For the same number of thrombin units (400 NIH Units), a higher quantity of Triblock ABA helps to decrease the blood flow (D1 vs D2) by absorbing the blood, by filling the entire wound and thus helping the diffusion of thrombin within the wound.
   b. Comparison of Prototypes E1 and E2

The results are summarized in FIG. 8. A comparison between the initial bleeding grade and the bleeding grades at 3 minutes and at 6 minutes is performed to evaluate the haemostatic performance of the prototypes tested.

The following observations can be made:
At 6 minutes, the blood flow remains the same when there is no haemostatic agent (Control Test).
At 6 minutes, the bleeding is stopped when prototypes E1 or E2 are introduced.
The prototypes containing calcium alginate (E2) seems to have a better performance than the prototypes containing CaCl2 (E1): the blood flow is stopped faster (no bleeding at 3 minutes for E2, while there is still a very slight bleeding at 3 minutes for E1).
The powder E1 (250 μm to 500 μm) has not the same granulometry as powder E2 (125 μm to 250 μm): the granulometry might have an influence on the bleeding reduction, we can assume that the smaller the granulometry is, the larger the contact surface.

### Example 6: Evaluation of the Effect of the Granulometry of the Powder According to the Invention

a. Material and Method

### Granulometry Measurement

The granulometry of the triblock is determined using sieves: the triblock powder is divided by grain size categories using a vibratory column sieve. The fractions corresponding to different particle sizes are separated and used to manufacture the prototype powders.

### Evaluation of the Expulsion Difficulty

A 500 mg sample of powder prototype has been introduced into a tube of 4.5 mm internal diameter: the operator performing the test will evaluate the difficulty to expel the powder from the tube using a pusher (tube) of 4.2 external diameter.

The difficulty scale is the following:
Easy: The powder is expelled easily from the tube, without using any effort.
Medium: The powder is expelled from the tube, using a slight effort.
Hard: The powder is expelled from the tube, using a big effort.
Impossible: The powder cannot be expelled from the tube.

The duration of the insertion procedure is measured too.

### Prototypes Tested

The powder prototypes C1-C2 and E1-E2 used for the in-vitro tests have been prepared using the method described in Example 1. The powder composition is detailed in Table 1 included in Example 1.

The powder prototypes E1 and C2 have been prepared using triblock of granulometry included between 250 μm and 500 μm, while the powder prototypes E2 and C1 have been prepared using triblock of granulometry included between 125 μm and 250 μm.

b. Results

The results of the expulsion test are detailed in the Table 3 below:

TABLE 3

| | E1 | C2 | E2 | C1 |
|---|---|---|---|---|
| Granulometry of the triblock (μm) | 250 < X < 500 | 250 < X < 500 | 125 < X < 250 | 125 < X < 250 |
| Expulsion duration (sec) | 27 | 8 | NA | 17 |
| Expulsion difficulty | Hard | Easy | Impossible | Medium |

The following observations can be made:
Without magnesium stearate (E1 and E2), the expulsion of the powder from the tube of 4.5 mm internal diameter is more difficult than with (see C2 and C1): the lubricant makes the powder easier to expel.
The higher the granulometry is (E1, C2), the easier is the expulsion from the tube. We can assume that the powder compacts much more with small grains than with large grains.

### Example 7: Evaluation of the Effect of the Lubricants on the Haemostatic Properties (In Vitro)

a. Material and Method
Hemostasis support: (see example 4)
In-vitro model: (see example 4)
Determination of clotting time: (see example 4)

### Prototypes Tested

The powder prototypes G0-G6 used for the in-vitro tests have been prepared using the method described in Example 1. The powder composition is detailed in Table 1 included in Example 1. The granulometry of the triblock (included in the powder prototypes) is between 250 μm and 500 μm.

b. Results

FIG. 9 presents the clotting time (min) as a function of the prototypes tested. Prototypes include different lubricants mixed with triblock and calcium alginate.

The following observations can be made:
It can be observed that the blood clotting time is shorter when the blood is in contact with the prototypes tested, in comparison with whole blood only.
The clotting time is the same for every prototype tested, regardless of the lubricant added: we can conclude that the type and quantity of lubricant added has no impact on the clotting time

### Example 8: Evaluation of the Effect of the Granulometry of the Powder and of the Lubricant Type

a. Material and Method
Granulometry measurement: see Example 6
Evaluation of the expulsion difficulty:

A 500 mg sample of powder prototype has been introduced into a tube of 4.6 mm internal diameter: the operator performing the test will evaluate the difficulty to expel the powder from the tube using a pusher (solid tube) of 4.5 mm external diameter.

The difficulty scale is the following:
Easy: The powder is expelled easily from the tube, without using any effort.
Medium: The powder is expelled from the tube, using a slight effort.
Hard: The powder is expelled from the tube, using a big effort.
Impossible: The powder cannot be expelled from the tube.

The duration of the insertion procedure is measured too.

### Prototypes Tested

The powder prototypes G0-G6 have been prepared using the method described in Example 1. The powder composition is detailed in Table 1 included in Example 1.

We will distinguish the granulometry of the powder preparation using particle-a for a granulometry between 125 μm and less than 250 μm, and particle-b for a granulometry between 250 μm and 500 μm (limits included).

G0-a to G6-a have been prepared using triblock of granulometry between 125 μm and less than 250 μm, while powder prototypes G0-b to G6-b have been prepared using triblock of granulometry between 250 μm and 500 μm (limits included).

Note: Only the triclock granulometry differentiate the prototypes G0-a and G0-b (as well as the other samples G1 to G6), their composition is the same.

b. Results

The results of the expulsion test are detailed in Table 4 below:

TABLE 4

| | Granulometry | | | |
|---|---|---|---|---|
| | of the | Lubricant | Expulsion | |
| Tested | triblock | type and | duration | Expulsion |
| prototypes | (μm) | percentage | (sec) | difficulty |
| G0-a | 125 ≤ X < 250 | / | 10 | Medium |
| G1-a | | 1% Stearate | 7 | Easy |
| | | Magnesium | | |
| G2-a | | 1% Sodium | 7 | Easy |
| | | Stearyl Fumarate | | |
| G3-a | | 1% Talc | 7 | Easy |
| G4-a | | 3% Stearate | 4 | Easy |
| | | Magnesium | | |
| G5-a | | 3% Sodium | 4 | Easy |
| | | Stearyl Fumarate | | |
| G6-a | | 3% Talc | 4 | Easy |
| G0-b | 250 ≤ X ≤ 500 | / | 7 | Medium |
| G1-b | | 1% Stearate | 4 | Easy |
| | | Magnesium | | |
| G2-b | | 1% Sodium | 4 | Easy |
| | | Stearyl Fumarate | | |
| G3-b | | 1% Talc | 4 | Easy |
| G4-b | | 3% Stearate | 3 | Easy |
| | | Magnesium | | |
| G5-b | | 3% Sodium | 3 | Easy |
| | | Stearyl Fumarate | | |
| G6-b | | 3% Talc | 3 | Easy |

The following observations can be made:
Without lubricant (G0-a and G0-b), the expulsion of the powder from the tube of 4.5 mm internal diameter is more difficult than with (G1-a to G6-a and G1-b to G6-b): the lubricant makes the powder easier to expel.
The higher the granulometry is (G0-b to G6-b), the faster is the expulsion from the tube.
With the same lubricant's content, no differences were observed between the different type of lubricants: their properties are identical and allow the powder to be easily expelled from the tube (see G1-G3 and G4-G6).
The more lubricant there is, the faster the powder can be expelled from the tube (see G4-G6 vs G1 to G3).
The granulometry of the triblock and the percentage of lubricant are two factors influencing the easiness and swiftness of powder expulsion.

1.-21. (canceled)

22. A powder composition comprising:
a degradable A and B block copolymer, wherein:
the A block is a polyester;
the B block is a poly(oxyethylene) (PEO);
the weight-average molecular mass of the blocks B is greater than or equal to 50 kDa; and
the ethylene oxide unit/ester unit mole ratio is between 0.5 and 5;
optionally, at least one lubricant; and
at least one haemostatic agent.

23. The powder composition according to claim 22, wherein the degradable A and B block copolymer has a particle size from 50 μm to 500 μm, as measured by sieving.

24. The powder composition according to claim 22, wherein the degradable A and B block copolymer has a particle size from 120 μm to 500 μm, as measured by sieving.

25. The powder composition according to claim 22, wherein the powder composition is free of lubricant.

26. The powder composition according to claim 22, wherein the haemostatic agent is selected from the group consisting of thrombin, calcium alginate, carboxymethylated starch, calcium salts, carboxymethylcellulose, gelatin, calcium ions, tranexamic acid, collagen, fibrinogen, and oxidized cellulose.

27. The powder composition according to claim 22, wherein the degradable A and B block copolymer is selected from AB diblock copolymers and ABA and BAB triblock copolymers, and mixtures thereof.

28. The powder composition according to claim 22, wherein the weight-average molecular mass of the blocks B in the A and B block copolymer is between 75 kDa and 150 kDa.

29. The powder composition according to claim 22, wherein the ethylene oxide unit/ester unit ratio of the A and B block copolymer is from 1 to 3.

30. The powder composition according to claim 22, wherein the blocks A of the A and B block copolymer are poly(lactic acids).

31. The powder composition according to claim 30, wherein the poly(lactic acids) are selected from the group consisting of poly(L-lactic acid), poly(D-lactic acid) and poly(D,L-lactic acid).

32. The powder composition according to claim 22, said powder composition comprising from 59% to 99.94% of the degradable A and B block copolymer, by weight according to the total weight of the powder composition.

33. The powder composition according to claim 22, said powder composition further comprising a carrier for the haemostatic agent.

34. The powder composition according to claim 33, wherein the carrier for the haemostatic agent is selected from the group consisting of lactose, carboxymethylated starch, polyethylene glycol (PEG), poly(oxyethylene) (PEO), and carboxymethylcellulose.

35. The powder composition according to claim 33, wherein the carrier for the haemostatic agent has a content from 8% to 30%, by weight according to the total weight of the powder composition.

36. The powder composition according to claim 22, wherein the powder composition comprises a lubricant.

37. The powder composition according to claim 36, wherein the lubricant is selected from the group consisting of magnesium stearate, stearic acid, sodium stearylfumarate, micronized polyoxyethyleneglycol, leucine, talc, sodium benzoate, and mixtures thereof.

38. The powder composition according to claim 36 comprising from 0.5% to 5% of the lubricant, by weight according to the total weight of the powder composition.

39. A method for preventing and/or treating haemorrhage in a body cavity of a subject comprising administering the powder composition according to claim 22 into the body cavity to be treated, in the subject in need thereof.

40. A method of preventing and/or treating uterine haemorrhage in a subject in need thereof, comprising administering the powder composition according to claim 22 into the uterine cavity of the subject in need thereof.

41. Kit comprising
(i) the powder composition according to claim 22; and
(ii) means for inserting said powder composition into a body cavity.
